(19) United States
(12) Patent Application Publication
(10) Pub. No.: US 2022/0400637 A1
(43) Pub. Date: Dec. 22, 2022

(54) SYSTEMS AND METHODS FOR LIQUID-MEDIATED DELIVERY OF POLLEN

(71) Applicant: Monsanto Technology LLC, St. Louis, MO (US)

(72) Inventors: Eric L. Borrowman, St. Peters, MO (US); David A. Morgenstern, Creve Coeur, MO (US)

(21) Appl. No.: 17/680,791

(22) Filed: Feb. 25, 2022

Related U.S. Application Data
(60) Provisional application No. 63/158,330, filed on Mar. 8, 2021.

(51) Int. Cl. A01H 1/02; B05B 7/12; B05B 7/24

(52) U.S. Cl. CPC A01H 1/027; B05B 7/1218; B05B 7/24

(57) ABSTRACT

The invention provides novel systems and methods for liquid-mediated delivery of pollen to a female reproductive part of a recipient plant. The systems provided herein include a container configured to receive a liquid pollen suspension solution, and an applicator attached to the container configured to spray the liquid pollen suspension solution onto the recipient plant. The methods provided herein include spraying a liquid pollen suspension solution onto at least a first female reproductive part of a recipient plant using the system provided herein, thereby pollinating the recipient plant.

### REFERENCE TO RELATED APPLICATION

This application claims the benefit of U.s. Provisional Application No. 63/158,330, filed Mar. 8, 2021, which is herein incorporated by reference in its entirety.

### FIELD OF THE INVENTION

The present disclosure relates to the field of agricultural biotechnology, and more specifically to systems and methods for improving pollination efficiency via liquid-mediated delivery of donor plant pollen grains to a female reproductive part of a recipient plant.

### BACKGROUND OF THE INVENTION

Cross-pollination is used in plant breeding to introduce hybrid vigor, new traits, and novel phenotypes, and is used as the first step in the breeding cycle for many crop plants. Conventional methods for cross-pollination in many crop species, such as corn (Zea mays, also known as maize), involves conventional pollination, which includes selective detasseling of female plants and interspersing rows of the male parent line in a field of the female parent line. This process is inefficient as it depends on the effective flow of pollen to the female plants, which is vulnerable to wind and other variables, and requires that the male and female plants enter the reproductive phase at the same time. In addition, selective detasseling of female plants is time consuming and labor-intensive, and male plants occupy field space but do not produce hybrid seed. Typical commercial breeding programs require thousands or even millions of crosses such as, development crosses, backcrosses, and crosses for trait integration. As breeders aim to accelerate crop variety development and reduce labor needs, it is critical to develop pollination methods that improve efficiency. Hybrid seed production, in particular, would greatly benefit from production methods that use fields consisting mostly or entirely of female plants.

### SUMMARY

In one aspect, the present disclosure provides a system for liquid-mediated delivery of pollen to a recipient plant, the system comprising: a container configured to receive a liquid pollen suspension solution; and an applicator attached to the container configured to spray the liquid pollen suspension solution onto the recipient plant. In one embodiment, the container comprises a bottom end and a top end, the bottom end comprising an opening configured to permit transfer of the liquid pollen suspension solution from the container to the applicator. In another embodiment, the container is further defined as a tube, a tank, or a basin. In yet another embodiment, the container is comprised of a substantially rigid material. Non-limiting examples of such substantially rigid materials include plastic, wood, metal, glass, and synthetic polymer. In still yet another embodiment, the container comprises an inner surface and an outer surface, the inner surface comprising at least one indentation or baffle.

In still yet another non-limiting embodiment, an applicator used in accordance with the invention is selected from the group consisting of an agricultural nozzle, a hydraulic liquid atomizing nozzle, and an air-assisted nozzle. In one embodiment, the applicator is configured to spray the liquid pollen suspension solution with a gas pressure of between about 5 psi and about 30 psi. In another embodiment, the applicator is configured to spray the liquid pollen suspension solution with an exit velocity between about 1 m/s and about 10 m/s. In yet another embodiment, the applicator is configured to produce droplets with a volume weighted mean droplet diameter of less than about 300 μm. In still yet another embodiment, the system comprises a receptacle attached to the container configured to maintain dry pollen at a preferred temperature. In one embodiment, the preferred temperature is between about 0.5° C. and about 10° C. In another embodiment, the system comprises a conveyor attached to the receptacle configured to facilitate the transfer of the dry pollen to the container, wherein the container comprises an liquid medium. In yet another embodiment, the system comprises a line configured to transfer the liquid pollen suspension solution to the applicator, the line comprising a first end and a second end, wherein the first end is connected to the container and the second end is connected to the applicator. The transfer may be facilitated, for example, by gravity, positive pressure, siphon feeding, a positive displacement pump, a centrifugal pump, or a peristaltic pump. In still yet another embodiment, the container comprises an agitator configured to mix the liquid pollen suspension solution. Non-limiting examples of agitators include a paddle stirrer, a rotating agitator, and a downward pumping impeller. In one embodiment, the system is configured to be mounted on a base to facilitate transport through a row of crop plants. In another embodiment, the system comprises a guide head configured to position a plant in an upright position in front of the applicator. In yet another embodiment, the system comprises at least one camera configured to obtain at least one image of at least one plant. In still yet another embodiment, the camera is in electronic communication with a processor configured to identify a location of a female reproductive part of the plant and transmit the location. In one embodiment, the applicator is configured to direct the spray of the liquid pollen suspension solution toward the location. In another embodiment, the applicator is attached to a repositioning assembly configured to position the applicator in response to receiving the location. In yet another embodiment, the applicator comprises a plurality of outlets. In still yet another embodiment, the applicator is configured to variably regulate a flow of the pollen suspension solution from the plurality outlets to direct the spray of the liquid pollen suspension solution toward the location. In one embodiment, the system comprises a plurality of applicators configured to spray the liquid pollen suspension solution onto the recipient plant.

In yet another illustrative embodiment, a system described herein may comprise at least one camera in electronic communication with a processor configured to (i) identify a location of a female reproductive part of the at least one plant; and (ii) transmit a location signal to a reception unit in response to identifying the location. In another embodiment the reception unit is configured to (i) receive the location signal from the identification unit; and (ii) cause at least one applicator from the plurality of applicators to direct the spray of the liquid pollen suspension solution toward the female reproductive part of the at least one plant in response to receiving the location signal. In still yet another embodiment, the pollen is from a monocot plant or is recalcitrant pollen. In one embodiment, the pollen is from a cereal plant, non-limiting examples of which include a corn, rice, wheat, or sorghum plant. In another embodiment, the system comprises chamber attached to the container configured to store a liquid medium. In yet another embodiment, the system comprises a conduit configured to facilitate the transfer of the liquid medium to the container, the conduit comprising a first end and a second end, wherein the first end is connected to the container and the second end is connected to the chamber. In still yet another embodiment, the transfer is facilitated by gravity, positive pressure, siphon feeding, a positive displacement pump, a centrifugal pump, or a peristaltic pump.

In another aspect, the present disclosure provides a method for liquid-mediated delivery of pollen to a recipient plant, the method comprising: (a) providing a system for liquid-mediated delivery of pollen to a recipient plant, the system comprising: (i) a container comprising a liquid pollen suspension solution; and (ii) an applicator attached to the container configured to spray the liquid pollen suspension solution onto the recipient plant; and (b) spraying the liquid pollen suspension solution onto at least a first female reproductive part of the recipient plant using the system, thereby pollinating the recipient plant. In one embodiment, the pollen is from a monocot plant or is recalcitrant pollen. In another embodiment, the pollen is from a cereal plant, non-limiting examples of which include a corn, rice, wheat, or sorghum plant. In some embodiments, the liquid pollen suspension is produced less than about 1 hour, less than about 20 minutes, less than about 5 minutes prior, or less than about 30 seconds prior to the spraying. In yet another embodiment, the spraying comprises spraying the liquid pollen suspension solution with a gas pressure of between about 5 psi and about 30 psi. In still yet another embodiment, the spraying comprises spraying the liquid pollen suspension solution with an exit velocity of between about 1 m/s and about 10 m/s. In one embodiment, the spraying produces droplets with a volume weighted mean droplet diameter of less than about 300 μm. In another embodiment, the method comprises repeating the steps of a) providing a system for liquid-mediated delivery of pollen to a recipient plant, the system comprising: (i) a container comprising a liquid pollen suspension solution; and (ii) an applicator attached to the container configured to spray the liquid pollen suspension solution onto the recipient plant; and b) praying the liquid pollen suspension solution onto at least a first female reproductive part of the recipient plant using the system, thereby pollinating the recipient plant on two or more consecutive days. In yet another embodiment, the spraying comprises air-assisted spraying. In still yet another embodiment, the method produces a substantially equivalent number of seeds compared to the number of seeds produced using a conventional pollination technique. In one embodiment, the method comprises collecting seed resulting from the pollinating. In another embodiment, the method comprises crossing a progeny plant grown from the seed with itself or a second plant. In yet another embodiment, the method comprises agitating the liquid pollen suspension prior to or concurrently with the spraying. The agitating may comprise for example mechanically moving the container or sparging the pollen suspension solution with a gas. In one embodiment, the recipient plant is male sterile at the time of the pollinating.

### BRIEF DESCRIPTION OF DRAWINGS

Those of skill in the art will understand that the drawings, described below, are for illustrative purposes only. The drawings are not intended to limit the scope of the present teachings in any way.

FIG. 1(a-c) shows a diagram of one embodiment of an applicator comprising a plurality of outlets from which the flow of the pollen suspension solution may be variably regulated.

FIG. 2 shows a frontal view of one embodiment of the present disclosure comprising a container, an applicator, a receptacle, a conveyor, a first pump, a chamber, a second pump, and an air compressor.

### DETAILED DESCRIPTION

Mechanical application of monocot pollen at the scale required for hybrid seed production has previously been impractical. Methods of spraying powder pollen are similarly limited in that monocot pollen clumps within hours of collection. Furthermore, methods for liquid-mediated delivery of pollen were previously ineffective due to pollen clumping and lysis upon exposure to water. A liquid-mediated delivery method was developed and is described herein to overcome these challenges and to provide for significantly improved delivery of monocot pollen to a female reproductive part of a recipient plant.

Modern plant breeding relies on outcrossing or cross-pollination to generate progeny plants having specific heritable traits. Such breeding strategies play an important role in F₁ population development and trait integration. Corn (Zea mays), rice (Oryza sativa), wheat (Triticum aestivum), and sorghum (Sorghum bicolor), which belong to the Poaceae family and the Liliopsida class (monocots) of plants, are examples of economically important agricultural crops in which breeding has been hampered by low efficiency procedures in controlled cross-pollination. Pollen of plants from the Poaceae family is classified as recalcitrant or desiccation sensitive as described in Pacini and Dolferus (Frontiers in Plant Sci. 10:679; 2019), specifically incorporated herein by reference. Other non-limiting examples of recalcitrant pollen include pollen of certain species in the Alismataceae, Amaranthaceae, Cactaceae, Chenopodiaceae, Cucurbitaceae, Anacardiaceae, Portulacaceae, Urticaceae, Lauraceae, Liliaceae, Iridaceae, Orchidaceae, Acanthaceae, and Caryophyllaceae families (Pacini and Dolferus, 2019). Conventional methods for cross pollination of such species, for example corn, entails emasculation of female plants and interspersing rows of male parent plants. This process is inefficient as it depends on the effective flow of pollen to the female plants, which is vulnerable to wind and requires that the male and female plants enter the reproductive phase at the same time.

Commercial production of hybrid seed is further constrained by the fact that salable seed is obtained only from male-sterile, female plants. The male parent of the hybrid, which provides pollen, represents a portion of the production cost. Any seed produced by male plants is inbred and cannot be marketed as hybrid seed. Thus, a method for artificial pollination which minimizes the pollen required, and thus the number of male plants required, is economically preferred. Using this criterion, the preferred method of pollination is manual application of a small amount of pollen directly to the silks of the female plant. In corn plants, full ears can typically be achieved with a single application of 32 mg of fresh pollen. While manual application is suitable for certain research purposes, such as plant breeding, it is too time consuming and labor-intensive for commercial production of hybrid seed. Therefore hybrid corn seed is produced in fields with male rows interspersed with female rows, relying on wind to transfer the pollen in commercial practice. Similarly inefficient methods are used for commercial production of hybrid rice, where a rope is used to bend male plants toward rows of female plants. A method to selectively spray pollen collected from male plants onto the female plants would allow for significantly more efficient pollen utilization, minimize resources for male plants, and would not be unacceptably labor-intensive provided that the spray can be applied while moving down the row at a reasonable speed.

The efficacy of pollen delivery systems and methods can be can be evaluated on the basis of several criteria. First, the system should singulate pollen so that it may be delivered to the female reproductive part of a recipient plant as individual pollen grains rather than as clumps. Delivery of singulated pollen promotes higher seed set. Secondly, the delivery system should deliver the pollen at a low spray velocity. Low spray velocity is critical for pollen retention on corn, wheat, and rice silks To promote efficient cross-pollination it may be desired to utilize systems and methods that direct as much pollen as possible toward the female reproductive part of a recipient plant. Liquid-mediated delivery systems are particularly equipped to direct singulated pollen toward the female reproductive part of a recipient plant at low spray velocity compared to spraying pollen in air. Air is a low-mass, low-viscosity carrier for pollen, which results in the onset of turbulence and loss of targeting potential in the spray pattern. Provided herein are systems and methods for liquid-mediated pollen delivery of pollen. Pollen suspension solutions for use in the systems and methods provided herein may be desired to be minimally phytotoxic towards both the pollen and the female reproductive part of the recipient plant.

The present invention represents a significant advance in the art in that it permits mechanical application of pollen to an all-female field, eliminating the need for in-field synchronized male and female plant development, and minimizing the effects of weather conditions. Application of monocot pollen at the scale required for hybrid seed production has previously been unfeasible. The pollen clumps within hours of collection, which makes it difficult to effectively spray powder pollen. Furthermore, the pollen rapidly becomes non-viable in water or when exposed to typical ambient environmental conditions. The current invention surprisingly overcomes limitations in the art by permitting cross-pollination using liquid-mediated delivery of pollen to a female reproductive part of a recipient plant, resulting in more efficient field use, eliminating the need for in-field synchronized male and female plant development, and minimizing the effects of variable weather conditions.

The present disclosure therefore permits implementation of high-throughput methods for the delivery of donor pollen to a recipient plant. The methods provided herein substantially reduce the time and labor previously required to facilitate cross-pollination. This is of particular significance as modern plant breeding programs may require thousands or even millions of individual crosses on a yearly basis in order to produce new plant varieties with improved traits.

### Systems and Methods for Liquid-Mediated Pollen Delivery

The present disclosure provides an integrated system for liquid-mediated pollen delivery that comprises a container configured to receive a liquid pollen suspension solution; and an applicator attached to the container configured to spray the liquid pollen suspension solution onto the recipient plant. In one embodiment, the container comprises a bottom end and a top end, the bottom end comprising an opening configured to permit transfer of the liquid pollen suspension solution from the container to the applicator. A “container” as used herein refers to a vessel capable of containing a liquid pollen suspension solution and providing for ingress and egress of the pollen suspension solution. The container may be of any appropriate geometrical shape, non-limiting examples of which include a cylinder, a sphere, a triangular prism, a cube, and a cone. The container may be, for example, a tube, a tank, or a basin. As used herein a “tube” refers to a long, hollow cylinder used for holding or transporting a substance. A “tank” as used herein refers to a large vessel or storage chamber. A “basin” as used herein refers to a wide, open container. In one embodiment, the container is comprised of a substantially rigid material, non-limiting examples of which include plastic, wood, metal, glass, and synthetic polymer. In another embodiment, the inner surface of the container may comprise at least one indentation or baffle. Indentations and baffles may find use in improving fluidization and suspension of pollen by creating local vortices that combat settling and pollen agglomeration. The term “about” is used to indicate that a value includes the standard deviation of the mean for the device or method being employed to determine the value.

As used herein, “pollen” refers to at least one pollen grain and may comprise a plurality of pollen grains. Non-limiting examples of pollen that may be used according to the system and methods of the invention include recalcitrant pollen, pollen collected from a dicot plant, a monocot plant, a cereal plant, a Poaceae family plant, an Alismataceae family plant, an Amaranthaceae family plant, a Cactaceae family plant, a Chenopodiaceae family plant, a Cucurbitaceae family plant, a Anacardiaceae family plant, a Portulacaceae family plant, a Urticaceae family plant, a Lauraceae family plant, a Liliaceae family plant, a Iridaceae family plant, a Orchidaceae family plant, a Acanthaceae family plant, a Caryophyllaceae family plant, a corn plant, a rice plant, a wheat plant, or a sorghum plant. As used herein “recalcitrant pollen” refers to desiccation sensitive pollen as described in Pacini and Dolferus (Frontiers in Plant Sci. 10:679; 2019). As used herein a “cereal plant” refers to grass plant cultivated for the edible components of its grain. Non-limiting examples of cereal plants include corn, rice, wheat, and sorghum plants. Pollen that may be used according to the systems and methods described herein includes any fertile pollen. Non-limiting examples of which include diploid pollen, double haploid pollen, transformed pollen, and pollen collected from transformed plants. Pollen for use in the present invention may be obtained using any manual or automated methods well known in the art. In certain embodiments, pollen may be fresh, or may be dried or partially dried, prior to being added to the system.

In one aspect, the present invention provides an applicator attached to the container configured to spray the liquid pollen suspension solution onto the recipient plant. The applicator forces liquid through a narrow opening to form the spray. Any applicator meeting this requirement can be used according to the systems and methods of the present invention to deliver a pollen suspension solution to a recipient plant. Non-limiting examples of applicators that may be utilized in the present invention include an agricultural nozzle, a hydraulic liquid atomizing nozzle, and an air-assisted nozzle. In some embodiments, applicators with relatively fine openings are used in order to provide optimal pollen singulation, examples of which include the TP series of nozzles from Teejet™. In another embodiment, the applicator may be an air-assisted applicator. Air-assisted applicators rely on an air stream to atomize the pollen suspension solution and thus can have relatively large openings. The air-assisted applicator may for example, have an opening with a diameter of about 0.02 inches to about 0.05 inches. Air-assisted applicators are well-known in the art and a number of designs are commercially available from a variety of manufacturers. One example of an air-assisted applicator is Paasche® LMR-1 airgun with a gravity-feed reservoir, which produces a flat-fan spray pattern useful for spraying rows of corn or other crops. Approximately one minute of spraying can be performed using the Paasche® LMR-1 airgun by adding the pollen suspension to the reservoir without risk of clogging. For continuous spray pollination, the reservoir can be replaced with a liquid line to the pump as described herein.

Other air-assisted applicators which, unlike the Paasche® LMR-1, are not usually operated as handheld applicators, produce a gentler, lower-velocity spray which enables improved targeting. Examples of this type of applicator include the EFX automatic airgun series from Graco®, which produces either a round or an oval spray pattern, which can be controlled by the choice of aircap. Atomization and spray velocity is controlled by the choice of air pressure. The use of the “airbrush” aircap produces a particularly fine spray in a round pattern, which may be desired for liquid-mediated delivery of aqueous pollen suspension solutions. An atomization air pressure of 5 psi provides acceptable atomization without excessive pollen velocity. Air-assisted applicators that are designed to spray at lower flow rates, such as the 1/8JJ Series from Spraying Systems Company, are effective at lower application rates of the pollen suspension solution. The 1/8JJ Series applicators produce either a round or flat spray pattern and the angle can be controlled by combining an aircap and a fluidcap to achieve the desired combination of flow rate and spray pattern. The fluid flow rate and air flow rate can be adjusted separately to optimize pollen suspension solution atomization, air speed, and application speed. Atomization can be achieved inside the nozzle (internal mix) or after the air and liquid exit the nozzle (external mix). When spraying a suspension solution that may be prone to clogging an additional clean out needle may be added to the applicator. This needle extends through the opening in the applicator to physically clear any blockages that may occur.

In one embodiment, the applicator is configured to spray the liquid pollen suspension solution with a gas pressure of between about 5 psi and about 30 psi. The applicator may for example spray the liquid pollen suspension solution with a gas pressure of about 5 psi, 10 psi, 15 psi, 20 psi, 25 psi, or 30 psi, including all ranges derivable therebetween. In another embodiment, the applicator is configured to spray the liquid pollen suspension solution with an exit velocity between about 1 m/s and about 10 m/s. The applicator may for example spray the liquid pollen suspension solution with an exit velocity of about 1 m/s, 2 m/s, 3 m/s, 4 m/s, 5 m/s, 6 m/s, 7 m/s, 8 m/s, 9 m/s, or 10 m/s, including all ranges derivable therebetween. In yet another embodiment, the applicator is configured to produce droplets with a volume weighted mean droplet diameter of less than about 300 μm. The applicator may for example produce droplets with a volume weighted mean droplet diameter of less than about 300 μm, 250 μm, 200 μm, 150 μm, or 100 μm, including all ranges derivable therebetween.

In one embodiment, the system comprises a receptacle attached to the container configured to maintain dry pollen at a preferred temperature. The preferred temperature may be for example between about 0.5° C. and about 10° C. The preferred temperature may be for example about 0.5° C., 1° C., 2° C., 3° C., 4° C., 5° C., 6° C., 7° C., 8° C., 9° C., or 10° C., including all ranges derivable therebetween. In another embodiment, the receptacle is configured to maintain the pollen at a preferred relative humidity. The preferred relative humidity may be for example between about 90% and about 100%. The preferred relative humidity may be for example about 90%, 91%, 92%, 93%, 94%, 95%, 96%, 97%, 98%, 99%, or 100%. In some embodiments, the receptacle may be configured to maintain pre-cooled pollen at a preferred temperature inside an insulated compartment or may be configured to maintain pollen at a preferred temperature by utilizing active refrigeration and climate control equipment known in the art. In another embodiment, the system comprises a conveyor attached to the receptacle configured to facilitate the transfer of the dry pollen to the container, wherein the container comprises an liquid medium. In yet another embodiment, the liquid medium inside the container is at ambient temperature. In still yet another embodiment, the liquid medium inside the container is an aqueous medium. In one embodiment, the system comprises chamber attached to the container configured to a store liquid medium. In another embodiment, the system comprises a conduit configured to facilitate the transfer of the liquid medium to the container, the conduit comprising a first end and a second end, wherein the first end is connected to the container and the second end is connected to the chamber. In still yet another embodiment, the transfer is facilitated by gravity, positive pressure, siphon feeding, a positive displacement pump, a centrifugal pump, or a peristaltic pump. In one embodiment, the liquid medium may be transferred directly into the container. For example the liquid medium may be poured into the container or transferred into the container using any method known in the art for transporting a liquid medium. In another embodiment, the pollen suspension solution may be produced outside the system and transferred directly into the container. The pollen suspension solution may be for example poured into the container or transferred into the container using any method of transporting a liquid medium known in the art.

Controlled addition of dry pollen to the liquid medium may be performed using any method which does not cause mechanical damage to the pollen. The controlled addition may be performed for example with an auger or screw conveyor. As described in U.S. Provisional App. Ser. No. 63/005,260, which is specifically incorporated herein by reference, pollen suspensions in aqueous media may be desired for liquid-mediated delivery provided that the contact time between the pollen and the aqueous medium is minimized. The contact time may for example maintain pollen viability and fertilization potential. In one embodiment, the contact time is less than about 5 minutes. In another embodiment, the contact time is less than about 2 minutes. In some embodiments, the pollen is present in the pollen suspension solution at about 2% to about 20% pollen by weight or at about 7 to about 12% pollen by weight. To minimize the contact time between the pollen and the aqueous medium, the pollen may be added to the aqueous medium continuously or in small batches with agitation to form and maintain a uniform pollen suspension solution. As described in U.S. Provisional App. Ser. No. 63/005,260, the addition high-molecular weight dispersants, such as METHOCEL™ modified cellulose polymers, facilitates pollen clump dispersal and aids in spray formation.

In one embodiment, the system comprises a line configured to transfer the liquid pollen suspension solution to the applicator, the line comprising a first end and a second end, wherein the first end is connected to the container and the second end is connected to the applicator. The transfer may be facilitated for example by gravity, positive pressure, siphon feeding, a positive displacement pump, a centrifugal pump, or a peristaltic pump. In some embodiments, pumps may provide better control over the rate of pollen application over a range of conditions, such as variable liquid rates and spray pressures. Pumps which avoid mechanical damage to pollen, and which are robust in the presence of suspended solids may be desired to convey the suspension from the container to the applicator. Peristaltic pumps avoid crushing the pollen and enable the accurate control of pollen flow through positive displacement. Centrifugal pumps pose minimal risk to pollen integrity and enable the control of pollen flow.

In another embodiment, the container comprises an agitator configured to mix the liquid pollen suspension solution. Non-limiting examples of agitators include a paddle stirrer, a rotating agitator, and a downward pumping impeller. Any method of agitation that maintains a uniform liquid pollen suspension and that does cause excessive pollen damage may also be used. Examples include vibrating, shaking, air mixing, vortexing, swirling, and continuously recirculating the liquid.

In yet another embodiment, the system is configured to be mounted on a base to facilitate transport through a row of crop plants. The equipment to facilitate transport of the system through a field or greenhouse may take different forms depending on the location and scale of the pollination. For small scale field or greenhouse pollinations, the system may be carried by the person performing the applications. The system may be for example mounted onto a backpack or cart. Larger scale operations require equipment that can navigate fields in the flowering stage. Motorized high clearance wheeled vehicles such as those sold by Hagie® are designed to travel over late stage corn fields for spraying. These vehicles are equipped with liquid storage equipment. Alternatively, unmanned aerial or ground vehicles could be utilized. These could be deployed as swarms of small scale vehicles or as larger individual vehicles with greater payload capacities.

In one embodiment, the system comprises a guide head configured to position a plant in an upright position in front of the applicator. Ear heights are relatively uniform in inbred female corn fields, however, the size of the ears can vary as does their orientation on the stalk. The plants can lean, be entangled with other plants, or sway in the wind. For these reasons, when spraying from a vehicle, a two-tine guide ahead may be mounted to the vehicle to position the plant upright and hold it in a substantially consistent position in relation to the sprayer.

In another embodiment, the system comprises at least one camera configured to obtain at least one image of at least one plant. In some embodiments, lights are mounted on the system to allow for pollination at night or in low-light conditions. In yet another embodiment, the camera is in electronic communication with a processor configured to identify a location of a female reproductive part of the plant and transmit the location. Targeting may be facilitated by the use of one or more forward-looking cameras on the system coupled with image analysis. Image analysis must be relatively fast, since even at 3 miles per hour, roughly nine plants must be sprayed per second at a 6-inch plant spacing, which is typical in American cornfields. This requires high-throughput image recognition. High-throughput image recognition is feasible because a cornfield presents a relatively uniform and consistent backdrop and because the ear is strikingly different from the rest of the plant in color and shape. Typically high-throughput image recognition is achieved using an algorithm based on a neural network. The use of a YOLO algorithm (“You only look once”) may be used because of the very high speed with which it can identify a bounding box containing the ear to target the spray. Other machine vision techniques using the difference in the spectral response of the silks as compared to the rest of the plant may also provide sufficient information regarding silk location. In addition, the specific spectrum of the silks can be indicative of the receptiveness of the silks to pollination. The imaging system that can identify non-receptive silks and avoid spraying them. Information regarding the location of the silks is passed from the image recognition software to the targeting system using electronic communication.

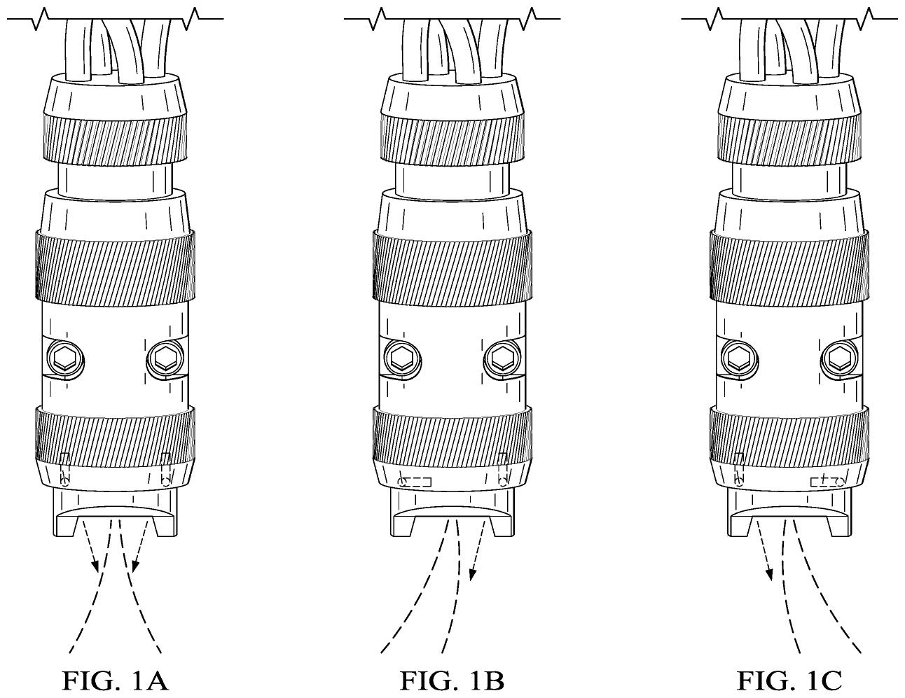

In yet another embodiment, the applicator is configured to variably regulate a flow of the pollen suspension solution from the plurality outlets to direct the spray of the liquid pollen suspension solution toward the location. FIG. 1 shows an applicator comprising a plurality of outlets from which the flow of the pollen suspension solution may be variably regulated. The gas pressure through each outlet may be regulated in manner that facilitates the direction of the flow toward the female recipient part of the plant. In some embodiments, the air pressure may be turned “on” to one or more outlets and may be turned “off” to one or more outlets in the plurality of outlets. In other embodiments, the gas pressure used to spray the pollen suspension solution may be varied for each outlet. Applicators designed to fluidized and shape the dispersion of a the pollen suspension solution may be used to deliver the solution to the female part of a recipient plant. Applicators that use an external mix design to combine gas and liquid may have multiple air outlets that function to atomize and shape the pattern of the dispersed liquid. By varying the amount of gas that traverses the applicator, the pattern of dispensed fluid can be altered without altering the orientation of the applicator. In addition, varying the relative amount of gas that exits each individual outlet can direct the pattern such that the solution is dispersed along a path that is not axial to the applicator. An applicator air cap designed with outlets positioned above, below, right, and left of the fluid outlet can be used to modify the fluid direction toward any point within a cone of limited angle. The ability of direct and aim the fluid flow without physically altering the applicator position may reduce the overall system complexity and increase the speed of the targeting.

In one embodiment, the applicator is configured to direct the spray of the liquid pollen suspension solution toward the location. In another embodiment, the applicator is attached to a repositioning assembly configured to position the applicator in response to receiving the location. Targeted spraying of the pollen for example may be accomplished with a gimballed one-axis or two-axis servo actuated nozzle. The coordinates of the silks relative to the equipment moving through the field along with the speed of the equipment informs the targeting system of the correct location to direct the pollen spray. High speed valves may then turn the flow of the pollen suspension solution on and off at the correct time to apply only the desired amount of pollen to each silk. In addition, the gimballed system may track the ear as spraying is occurring in order to increase the amount of time that is available for application to each ear.

In one embodiment, the system comprises a plurality of applicators configured to spray the liquid pollen suspension solution onto the recipient plant. In another embodiment, the at least one camera is in electronic communication with a processor configured to (i) identify a location of a female reproductive part of the at least one plant; and (ii) transmit a location signal to a reception unit in response to identifying the location. In yet another embodiment the reception unit is configured to (i) receive the location signal from the identification unit; and (ii) cause at least one applicator from the plurality of applicators to direct the spray of the liquid pollen suspension solution toward the female reproductive part of the at least one plant in response to receiving the location signal. As one example, a system using multiple applicators in either 1 or 2 axis arrays may be utilized. The silk location information from the image recognition software is used to turn “on” only the applicators that are in the correct position relative to the silks. In one embodiment, repositioning of the applicators is not required. In another embodiment, slower speed vertical actuation may be used to accommodate for more drastic variations in the overall height of the silks Such variability may be due to plant genetics or growing conditions within or between fields. Vertical actuation may be achieved utilizing feedback from the silk imaging system, feedback from an automatic plant height detection system, or manual adjustment by the operator.

The embodiments of the disclosure described herein are not intended to be exhaustive or to limit the disclosure to the precise forms disclosed. Instead, the embodiments selected for description have been chosen to enable one skilled in the art to practice the invention. It should be understood that the concepts presented herein may be used in various applications and should not be limited to use in the specific embodiments depicted in the drawings.

FIG. 1 is a diagram showing an applicator 101 comprising a first outlet 102 and a second outlet 103 from which the flow of the pollen suspension solution may be variably regulated. FIG. 1A shows a spray pattern that may be produced when the flow of the pollen suspension solution through both the first outlet 102 and the second outlet 103 is equal. FIG. 1B shows a spray pattern that may be produced when the flow of the pollen suspension solution is increased through the second outlet 103 compared to the flow of the pollen suspension solution through the first outlet 102. FIG. 1C shows a spray pattern that may be produced when the flow of the pollen suspension solution is increased through the first outlet 102 compared to the flow of the pollen suspension solution through the second outlet 103.

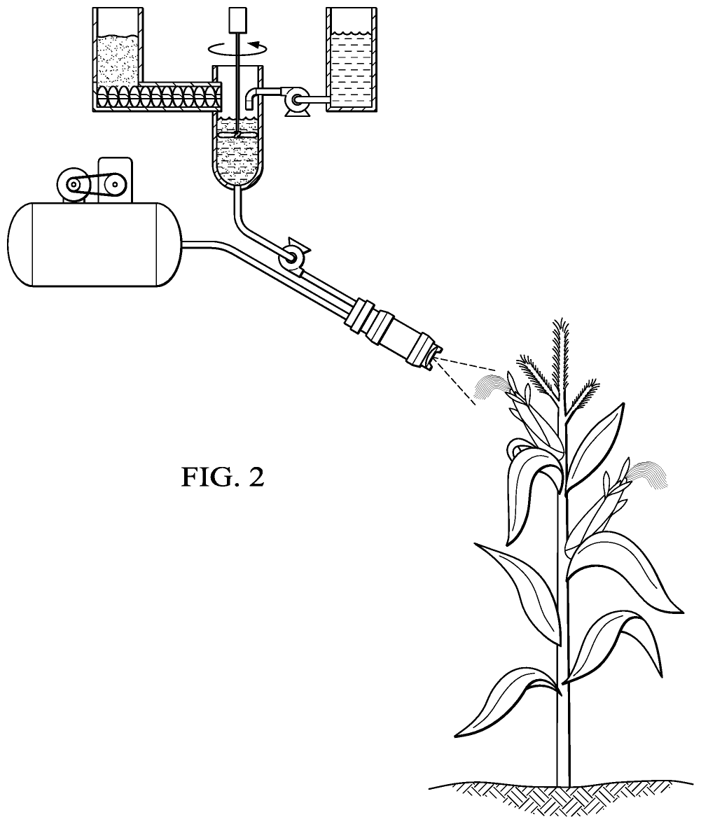

FIG. 2 is a diagram showing a system having a container 201 to receive a liquid pollen suspension solution; an applicator 202 attached to the container 201 to spray the liquid pollen suspension solution; a receptacle 203 attached to the container 201 to maintain dry pollen at a preferred temperature; a conveyor 204 attached to the receptacle 203 to facilitate the transfer of the dry pollen to the container 201; a line 205 comprising a first end and a second end, wherein the first end is connected to the container 201 and the second end is connected to the applicator 202 to facilitate the transfer of the liquid pollen suspension solution from the container 201 to the applicator 202; a pump 206 to facilitate the transfer along the line 205; an agitator 207 to mix the pollen suspension solution in the container 201; a chamber 208 attached to the container 201 to store a liquid medium; a conduit 209 comprising a first end and a second end, wherein the first end is connected to the container 201 and the second end is connected to the chamber 208 to facilitate the transfer of the liquid medium to the container 201; a pump 210 to facilitate the transfer of the liquid medium along the conduit 209; and an air compressor 211 attached to the applicator 202 to facilitate air-assisted spraying by the applicator 202.

The present invention surprisingly permits cross-pollination of potentially any flowering plant or grass using stored a liquid pollen suspension solution. In one embodiment, a method for pollinating a plant is provided herein, the method comprising: (a) providing a system for liquid-mediated delivery of pollen disclosed herein; and (b) spraying the liquid pollen suspension solution onto at least a first female reproductive part of the recipient plant using the system, thereby pollinating the recipient plant. As used herein the term “spraying” refers to generating droplets of a pollen suspension solution of a size capable of delivering pollen to female reproductive portions of a recipient plant, thereby pollinating the recipient plant. In some embodiments, the methods of the invention may be optimized for a particular application, particular plant species, or particular pollen type. Such parameters can be determined empirically using the methodology described herein. Non-limiting examples of plants that may be used according to the methods of the invention include plants with recalcitrant pollen, dicot plants, monocot plants, cereal plants, Poaceae family plants, Alismataceae family plants, Amaranthaceae family plants, Cactaceae family plants, Chenopodiaceae family plants, Cucurbitaceae family plants, Anacardiaceae family plants, Portulacaceae family plants, Urticaceae family plants, Lauraceae family plants, Liliaceae family plants, Iridaceae family plants, Orchidaceae family plants, Acanthaceae family plants, Caryophyllaceae family plants, corn plants, rice plants, wheat plants, and sorghum plants.

In some embodiments, the liquid pollen suspension is produced less than about 30 seconds, 1 minute, 2 minutes, 5 minutes, 10 minutes, 20 minutes, 30 minutes, 1 hour, or 2 hours prior to the spraying. In one embodiment, the spraying comprises spraying the liquid pollen suspension solution with a gas pressure of between about 5 psi and about 30 psi. The spraying may for example comprise spraying the liquid pollen suspension solution with a gas pressure of about 5 psi, 10 psi, 15 psi, 20 psi, 25 psi, or 30 psi, including all ranges derivable therebetween. In still yet another embodiment, the spraying comprises spraying the liquid pollen suspension solution with an exit velocity of between about 1 m/s and about 10 m/s. The spraying may comprise for example spraying the liquid pollen suspension solution with an air exit velocity of about 1 m/s, 2 m/s, 3 m/s, 4 m/s, 5 m/s, 6 m/s, 7 m/s, 8 m/s, 9 m/s, or 10 m/s, including all ranges derivable therebetween. In one embodiment, the spraying produces droplets with a volume weighted mean droplet diameter of less than about 300 μm. The spraying may for example produce droplets with a volume weighted mean droplet diameter of less than about 300 μm, 250 μm, 200 μm, 150 μm, or 100 μm, including all ranges derivable therebetween. In another embodiment, the method comprises repeating the steps of a) providing a system for liquid-mediated delivery of pollen disclosed herein; and b) praying the liquid pollen suspension solution onto at least a first female reproductive part of the recipient plant using the system, thereby pollinating the recipient plant on two or more consecutive days. These steps may be repeated, for example, on two consecutive days, three consecutive days, four consecutive days, or on five or more consecutive days. In corn, for example, it can be found that repeating the delivering steps on two or three consecutive days can result in higher seed set.

Spraying may include but is not limited to air-assisted spraying or spraying using a common agricultural nozzle. Air-assisted spraying relies on an air stream to atomize the pollen suspension solution. Air-assisted applicators for air-assisted spraying are well-known in the art and a number of designs are commercially available from a variety of manufacturers. Spraying using a common agricultural nozzle comprises forcing liquid through a narrow opening to form the spray. In one embodiment, the method produces a substantially equivalent number of seeds compared to the number of seeds produced using a conventional pollination technique. Substantial equivalence is evaluated by comparing seed sets produced using liquid-mediated pollen delivery to seed sets produced using one day hand pollination, where pollen from the same lot is applied to female plants from the same lot on the same day. As used herein, “substantially equivalent” refers to a characteristic wherein the mean value±standard deviation of the test population does not deviate more than about 20% from the mean value±standard deviation of the control population.

In yet another embodiment, the method comprises agitating the liquid pollen suspension prior to or concurrently with the spraying. The agitating may comprise for example mechanically moving the container or sparging the pollen suspension solution with a gas. Any method of agitation that maintains a uniform liquid pollen suspension and that does cause excessive pollen damage may also be used. Examples include vibrating, shaking, air mixing, vortexing, swirling, and continuously recirculating the liquid.

The step of collecting seed resulting from pollinating according to the systems and methods of the invention is provided herein. In a particular embodiment, a progeny plant produced from the collected seed may be crossed with itself or a different plant. In certain embodiments, a method of producing hybrid seed is provided herein comprising producing pollen, delivering the pollen to a female reproductive part of a recipient plant using the systems and methods described herein, thereby pollinating the female reproductive part with the pollen from the donor plant, harvesting seed produced from the pollination; and identifying hybrid progeny. Selecting a progeny seed or plant that results from pollinating may also performed. Identifying and selecting progeny could be facilitated by use of a polymorphic marker allele contained in the pollen donor that serves to identify progeny plants or seeds of that donor. Morphological markers or biochemical/protein markers have commonly been used as tools for selection of plants with desired traits in breeding. Molecular marker techniques that have been extensively used and are particularly promising for application to plant breeding include: restriction fragment length polymorphisms (RFLPs), amplified fragment length polymorphisms (AFLPs), random amplified polymorphic DNA (RAPD), microsatellites or simple sequence repeats (SSRs), and single nucleotide polymorphisms (SNPs) (Al-Khayri, et al., Advances in Plant Breeding Strategies, 2016).

In still other embodiments, the methods described herein may comprise pollination of flowers that are male sterile at the time of pollinating. Depending upon the developmental stage of the plant, donor pollen applied for cross-pollination could compete with pollen produced by the recipient plant. In order to improve the efficacy of the cross-pollination, it may be advantageous in some cases that the recipient plant be male sterile in an effort to reduce competition with selfing. Thus, a male sterility system could be employed with the female parent plant in a particular cross. Many such male sterility systems are well known, including cytoplasmic male sterility (CMS) and genic male sterility (GMS). CMS and GMS facilitate hybrid seed production for many crops and thus allow breeders to harness yield gains associated with hybrid vigor. The use of a gametocide presents an alternative method to produce male sterility. Gametocides affect processes or cells involved in the development, maturation or release of pollen. Plants treated with such gametocides are rendered male sterile, but typically remain female fertile. The use of chemical gametocides is described, for example, in U.S. Pat. No. 4,936,904, the disclosure of which is specifically incorporated herein by reference in its entirety. Furthermore, the use of Roundup herbicide in combination with glyphosate tolerant corn plants to produce male sterile corn plants is disclosed in PCT Publication WO 98/44140. Several gametocides have been reported effective in inducing pollen sterility in various crops and are well known in the art. Such gametocides include sodium methyl arsenate, 2,3-dichloroisobutyrate, sodium 2,2-dichloropropionate, gibberellic acid, maleic hydrazide (1,2-dihydropyridazine, 3-6-dione), 2,4-dichloro phenoxy acetic acid, ethyl 4-fluorooxanilate, trihalogenated methylsulfonamides, ethyl and methyl arsenates (Ali et al., Genetics Plant Breeding, 59:429-436, 1999). Physical emasculation of the recipient plant presents another alternative to produce male sterility. Following emasculation, the plants are then typically allowed to continue to grow and natural cross-pollination occurs as a result of the action of wind, which is normal in the pollination of grasses, including corn. As a result of the emasculation of the female parent plant, all the pollen from the male parent plant is available for pollination because the male reproductive portion, and thereby pollen bearing parts, have been previously removed from all plants of the plant being used as the female in the hybridization. Of course, during this hybridization procedure, the parental varieties are grown such that they are isolated from other plants to minimize or prevent any accidental contamination of pollen from foreign sources. These isolation techniques are well within the ability of those skilled in this art.

The methods disclosed herein may be implemented for improved cross-pollination of potentially any plants. Such plants can include, but are not limited to, cereal plants, non-limiting examples of which are corn, wheat, rice, and sorghum.

### Liquid Pollination Solution Formulations

The systems and methods described herein may be implemented to deliver potentially any liquid pollen suspension solution. Non-limiting examples of liquid pollen suspension solutions that may be delivered using the systems and methods provided herein are described in U.S. Provisional App. Ser. No. 63/005,260, which is incorporated herein by reference. In one embodiment the liquid pollen suspension solution may comprise a surfactant, an oil or an aqueous solution, and about 2% to about 20% pollen by weight. In some embodiments, the optimum components for use in the liquid pollen suspension solution may be optimized for a particular application. Such parameters can be determined empirically using the methodology described herein. To promote cross-pollination, for example, it may be desired to use a pollen suspension solution containing components that facilitate uniform pollen dispersal, maintain high viability of the pollen grains, and which do not significantly hinder fertilization and seed development when sprayed onto the female reproductive part of a recipient plant.

Non-limiting examples of components that may be used in the production of such a solution are provided herein and may include, in certain embodiments, an aqueous solution, an oil, a surfactant, an organic solvent, a disaccharide, or a polysaccharide. In some embodiments, the solution may be an aqueous solution or may be comprised in other solvents. In some embodiments, the solution may comprise an oil and an aqueous solution. In some embodiments, the solution may comprise an oil, which serves to facilitate long term cold storage and viability of the pollen. Embodiments of the invention may comprise any oil known in the art, including for example a paraffin, an isoparaffin, or a silicone oil, or any combination thereof. In some embodiments, the solution may comprise a synthetic solvent, for example Isopar M™, which may be in the solution at a concentration of about 48% to about 100% Isopar M™ by weight. In some embodiments, the solution may comprise a surfactant, which serves to uniformly disperse pollen in the solution. Embodiments of the invention may comprise any surfactant, or combination of surfactants, known in the art, for example modified cellulose polymer, a block copolymer of ethylene oxide and propylene oxide, or an agronomically acceptable dispersant polymer. In certain embodiments, the surfactant may be a block copolymer of ethylene oxide and propylene oxide further comprising a terminal alkyl group. In some embodiments, the surfactant may be one or more of Atlox™ LP-1, Lutensol® XL-80, Pluronic® P104, Walocel™ C CRT30, Poly Suga® Mulse, Mazol 300K, BREAK THRU® DA 647, TOXIMUL® 8325, Atlas G-5000, METHOCEL™ F50, Surfynol®, METHOCEL™ E19, BREAK THRU® DA 675, Atlox™ 4915, TOXIMUL® 8320, or TOXIMUL® 8242, and may, for example, be in the solution at a concentration of less than about 5.0%, less than about 4.0%, less than about 3.0%, less than about 2.0%, less than about 1.0%, or less than about 0.5% surfactant by weight. In a specific embodiment, the pollen suspension solution may comprise an aqueous solution of 10% pollen in 0.2% METHOCEL™ F50 which is agitated at ambient temperature.

In some embodiments, the solution may comprise a disaccharide or a polysaccharide, which serves to prevent pollen lysis in aqueous solution. In certain embodiments, the pollen is impermeable to the disaccharide or polysaccharide Embodiments of the invention may comprise any disaccharide or polysaccharide, or any combinations of disaccharides or polysaccharides, known in the art. A disaccharide or polysaccharide may be present in the pollen suspension solution at a concentration of about 5% to about 50% disaccharide or polysaccharide by weight.

### Modified Plants and Seeds

One aspect of the invention provides selection of progeny plants and seeds that result from the methods described herein. In some embodiments, the progeny plants and seeds may be defined as comprising a detectable modification relative to the female parent plant. One method of producing such plants and seeds is through use of an allele produced by plant genetic transformation. Suitable methods for transformation of host plant cells for use with the current invention are well known in the art and include any method by which DNA can be introduced into a cell (for example, where a recombinant DNA construct is stably integrated into a plant chromosome) and are well known in the art. Some widely utilized methods for cell transformation are Agrobacterium-mediated transformation, microprojectile bombardment-mediated transformation, and cell penetrating peptide-mediated delivery of DNA modifying agents.

Another method of producing modified plants and seeds is through genome editing. As used herein, the term “genome editing” refers to the use of genome editing methods and a site-specific genome modification enzyme to modify a nucleotide sequence. In some embodiments, donor pollen may be transformed using techniques known in the art to contain one or more reagents that mediate genome-specific modification in a plant. Pollen grains may be used in accordance with the invention that comprise any such reagents of loci generated with use of such reagents at any current or prior generation.

Suitable methods for altering a wild-type DNA sequence at a pre-determined chromosomal site include any method known in the art. Targeted modification of plant genomes through the use of genome editing methods and reagents can be used to create improved plant lines through modification of plant genomic DNA. In addition, genome editing methods and reagents can facilitate targeted insertion of one or more nucleic acids of interest into a plant genome. Exemplary methods for introducing donor polynucleotides into a plant genome or modifying the genomic DNA of a plant include the use of genome editing reagents such as: sequence-specific recombinases, endonucleases, zinc-finger nucleases, engineered or native meganucleases, TALE-endonucleases, RNA-guided endonucleases (for example, a Clustered Regularly Interspersed Short Palindromic Repeat (CRISPR)/Cas9 system, a CRISPR/Cpf1 system, a CRISPR/CasX system, a CRISPR/CasY system, a CRISPR/Cascade system), and CRISPR-associated transposases (Strecker, et al., Science, 365(6448):48-53, 2019) and (Klompe, et al., Nature, 571:219-225, 2019). Several embodiments relate to methods of genome editing using single-stranded oligonucleotides to introduce precise base pair modifications in a plant genome, as described by Sauer et al. (Plant Physiol. 170(4):1917-1928; 2016).

As used herein, the term “site-specific genome modification enzyme” refers to any enzyme that can modify a nucleotide sequence in a sequence-specific manner. In some embodiments, a site-specific genome modification enzyme modifies the genome by inducing a single-strand break. In some embodiments, a site-specific genome modification enzyme modifies the genome by inducing a double-strand break. In some embodiments, a site-specific genome modification enzyme comprises a cytidine deaminase. In some embodiments, a site-specific genome modification enzyme comprises an adenine deaminase. In the present disclosure, site-specific genome modification enzymes include endonucleases, recombinases, transposases, deaminases, helicases and any combination thereof. In some embodiments, the site-specific genome modification enzyme is a sequence-specific nuclease.

### EXAMPLE 1

### Liquid-Mediated Pollen Delivery in Corn Plants Using Oil Based Solutions

Liquid-mediated delivery of monocot pollen to a female reproductive part of a recipient monocot plant is challenging as monocot pollen has a tendency to clump and lyse upon exposure to water. A liquid-mediated delivery method was developed to overcome these challenges and to deliver monocot pollen to a female reproductive part of a recipient monocot plant with minimal water using oil based solutions.

A suitable liquid-mediated pollen delivery method was evaluated by examining seed set following pollination according to the following protocols: 1) 32 mg dry corn pollen, sprinkled; 2) 50 mg corn pollen in 300 mg IL3 (3.0% Atlox™ LP1 in Isopar M); 3) 50 mg corn pollen in 600 mg IL3; 4) 50 mg corn pollen in 300 mg M3 oil (Mazol 300K 0.7%; Atlox™ LP1 3.0%; Isopar M 96.3%)+300 mg 23% PEG1500/1.0% Pluronic® P104; 5) 50 mg corn pollen in 300 mg IL3+300 mg 2% Walocel™ C CRT30/1.0% Pluronic® P104. For the dry control, 32 mg of fresh pollen was weighed into vials that were kept cool until pollen was sprinkled onto the silks. For the other protocols, 50 mg of pollen was added to vials comprising the oil, which was pre-conditioned at 7° C., to make a pollen suspension solution. Pollen suspension solutions comprising oil or aqueous media were applied to ears using air-assisted spraying. The pollen suspension solutions were either vortexed and added immediately to the reservoir of a gravity-fed airbrush (Paasche® TG-3F from Paasche Airbrush, Kenosha, Wis.), or combined with an aqueous solution, shaken, and poured into the airbrush reservoir. Pollen suspension solutions were promptly sprayed directly over the ear using the airbrush and a 0.66 m or 0.38 mm tip at 20-35 psi. All pollinations were completed within 2.5 hours of adding pollen to oil. Oil pollen suspension solutions without emulsification showed a clear improvement in seed set when using 600 mg rather than 300 mg of oil (Table 1).

TABLE 1
Seed set following airbrush application of oil-based pollen
suspension solution with and without emulsification.

| Formulation | Ear 1 | Ear 2 | Ear 3 | Average |
|---|---|---|---|---|
| Dry, sprinkled | 209 | 317 | 372 | 299 ± 67.71 |
| 300 mg IL3 | 0 | 99 | N/A | 49.5 ± 49.5 |
| 600 mg IL3 | 98 | 228 | N/A | 163 ± 65 |
| M3/PEG1500 | 4 | 3 | 70 | 25.67 ± 31.35 |
| IL3/Walocel ™ | 0 | 1 | 7 | 2.67 ± 3.09 |

### EXAMPLE 2

### Liquid-Mediated Pollen Delivery in Corn Plants Using Aqueous Solutions

Liquid-mediated delivery of monocot pollen to a female reproductive part of a recipient monocot plant is challenging as monocot pollen has a tendency to clump and lyse upon exposure to water. A liquid-mediated delivery method was developed to overcome these challenges and to deliver monocot pollen to a female reproductive part of a recipient monocot plant with minimal water using aqueous solutions.

A liquid-mediated pollen delivery method using multiple applications of aqueous pollen suspension solutions and variable agitation methods was evaluated by examining seed set following pollination according to the following protocols: 1) 32 mg corn pollen, dry; 2) 40 mg corn pollen in tap water, sparge; 3) 40 mg corn pollen in tap water, vortex; 4) 40 mg corn pollen in 0.5% METHOCEL™ F50, sparge; 5) 40 mg corn pollen in 0.5% METHOCEL™ F50, vortex; 6) 40 mg corn pollen in 0.2% TOXIMUL® 8320, sparge; 7) 40 mg corn pollen in 0.2% TOXIMUL® 8320, vortex; 8) 40 mg corn pollen in 0.2% Atlox™ 4915, sparge. Corn pollen was added to the aqueous solution in either a vial and vortexed or in the reservoir of a Paasche™ TG-3F gravity-fed airbrush, and sparged immediately prior to spraying promptly over the ear using a 0.38 mm tip at 30 psi. All liquid-mediated delivery protocols were repeated for a total of three applications on three consecutive days. The ears were collected and evaluated 10 days after the first pollination. The three vortexed pollen suspension solutions produced ears that were visually full or nearly full (Table 2).

TABLE 2
Seed set following multiple airbrush applications
of aqueous pollen suspension solutions over consecutive
days using variable agitation methods.

| Protocol | Ear 1 | Ear 2 | Ear 3 | Average |
|---|---|---|---|---|
| Dry, sprinkled | 456 | 416 | 498 | 457 ± 41 |
| Tap water, sparge | 62 | 179 | 203 | 148 ± 75 |
| 0.5% METHOCEL ™ F50, sparge | 101 | 130 | 73 | 101 ± 28 |
| 0.2% TOXIMUL ® 8320, sparge | 199 | 169 | 131 | 166 ± 34 |
| 0.2% Atlox ™ 4915, sparge | 166 | 160 | — | 163 ± 4 |
| Tap water, vortex | 372 | 300 | 407 | 360 ± 55 |
| 0.5% METHOCEL ™ F50, vortex | 260 | 353 | 315 | 309 ± 47 |
| 0.2% TOXIMUL ® 8320, vortex | 294 | 277 | 346 | 306 ± 36 |

### EXAMPLE 3

### Liquid-Mediated Pollen Delivery to Corn Plants in Field and Greenhouse Conditions

A suitable liquid-mediated pollen delivery method was evaluated by examining seed set following pollination according to the following protocol. Corn pollen was mechanically collected from inbred male plants and stored in a cooler for several hours prior to production of a pollen suspension solution comprising 7.5 g of corn pollen and 68 ml of 0.2% METHOCEL™ F50. The solution was shaken and poured into the reservoir of a Paasche™ LMR-1 airgun with a gravity-feed reservoir, and sprayed while walking at 3 mph along one side of a 50-foot row of cytoplasmic male-sterile corn plants, which were spaced 6 feet apart. Three passes were made along each side of the row, corresponding to delivery of 150 mg pollen per plant. The liquid-mediated delivery protocol was repeated for a total of three applications on three consecutive days, resulting in total pollen delivery of 450 mg pollen per plant in each trial. Two trials using two different cytoplasmic male-sterile corn lines as the pollen recipient were completed using the above referenced protocol, and despite high fungal and insect pressure, seed sets of 76±44 and 156±60 kernels per ear were achieved. The typical seed set for the corn variety used in this trial is approximately 300 kernels per ear. Negative control plants produced only one seed per ear on average, demonstrating the near-perfect male sterility of the variety used in these trials.

A suitable liquid-mediated pollen delivery method was evaluated by examining seed set following pollination according to the following protocol. A row of six female plants were pollinated in a greenhouse using a pollen suspension solution comprising 10% corn pollen in 0.2% METHOCEL™ F50 and a Graco® EFX automatic airgun with an atomization air pressure of 5 psi. The six plants were sprayed in a continuous back-and-forth pass with the airgun using a feed of 10% pollen in 0.2% METHOCEL™ F50 pumped to the airgun at rate of 15 ml/min with a peristaltic pump. This produces a well-atomized, well-targeted spray at a comparatively low velocity, which improves pollen retention on the ears. In this trial the liquid-mediated delivery protocol was performed on only one day, but still produced a seed set of 201±117 kernels per ear. The typical seed set for the corn variety used in this trial is approximately 300 kernels per ear. This demonstrates that good seed set can be achieved with minimal pollen by means of well-directed, gentle air-assisted spraying of pollen in an aqueous medium.

A suitable liquid-mediated pollen delivery method was evaluated by examining seed set following pollination according to the following protocol. A row of five female plants were pollinated using a pollen suspension solution comprising 10% corn pollen in 0.2% METHOCEL™ F50 and a stainless steel Teejet® TP 6501 nozzle. This nozzle produces a particularly fine spray as a narrow 65-degree fan that can be accurately targeted. The solution was pumped at 270 ml/min through the nozzle, which was passed back and forth over the row of five plants for 5 seconds, resulting in the delivery of 450 mg pollen/plant. The protocol was repeated for a total of three applications on three consecutive days, and resulted in a seed set of 177±40 kernels per ear. This demonstrates that acceptable seed set can be achieved with conventional nozzles that produce a very fine spray.

### EXAMPLE 4

### Further Applications of the Novel Liquid-Mediated Pollen Delivery Method

Transgenic seeds or gene-edited seeds of recipient plants may be directly generated through liquid-mediated pollination with exogenous DNA-transformed pollen. Collected pollen may be transformed through physical methods such as electroporation, bombardment and sonication, Agrobacterium infection, pollen tube-mediated transfection, or magnetofection (Zhao, et al., 2017). For example, CRISPR/Cpf1 reagents may be delivered into purified pollen grains using electroporation or magnetofection. The transformed pollen is then selected and placed into a liquid solution provided herein. The pollen solution may then be sprayed onto the female reproductive portion of a recipient plant to create genome-edited seeds. It is feasible to utilize the liquid-mediated pollination methods provided herein and CRISPR/Cpf1-based gene editing for trait discovery and improvement in plants. This combination obviates the need for the laborious steps of tissue culture while producing transgenic or gene-edited plants from transformed seeds within a short period of time.

What is claimed is:

1. A system for liquid-mediated delivery of pollen to a recipient plant, the system comprising:
a container configured to receive a liquid pollen suspension solution; and
an applicator attached to the container configured to spray the liquid pollen suspension solution onto the recipient plant.

2. The system of claim 1, wherein:
a) the container comprises a bottom end and a top end, the bottom end comprising an opening configured to permit transfer of the liquid pollen suspension solution from the container to the applicator;
b) the container is further defined as a tube, a tank, or a basin;
c) the container is comprised of a substantially rigid material;
d) the container comprises an agitator configured to mix the liquid pollen suspension solution;
e) the applicator is selected from the group consisting of an agricultural nozzle, a hydraulic liquid atomizing nozzle, and an air-assisted nozzle;
f) the applicator is configured to spray the liquid pollen suspension solution with a gas pressure of between about 5 psi and about 30 psi;
g) the applicator is configured to spray the liquid pollen suspension solution with an exit velocity between about 1 m/s and about 10 m/s;
h) the applicator is configured to produce droplets with a volume weighted mean droplet diameter of less than about 300 μm;
i) the system is configured to be mounted on a base to facilitate transport through a row of crop plants;
j) the system comprises a plurality of applicators configured to spray the liquid pollen suspension solution onto the recipient plant;
k) the system comprises a receptacle attached to the container configured to maintain dry pollen at a preferred temperature;
l) the system comprises at least one camera configured to obtain at least one image of at least one plant;
m) the system comprises a line configured to transfer the liquid pollen suspension solution to the applicator, the line comprising a first end and a second end, wherein the first end is connected to the container and the second end is connected to the applicator; or
n) the pollen is:
A) from a monocot plant; or
B) recalcitrant pollen.

3. The system of claim 2, wherein:
a) the substantially rigid material is selected from the group consisting of plastic, wood, metal, glass, and synthetic polymer;
b) the container comprises an inner surface and an outer surface, the inner surface comprising at least one indentation or baffle;
c) the agitator is a paddle stirrer, a rotating agitator, or a downward pumping impeller;
d) said at least one camera is in electronic communication with a processor configured to identify a location of a female reproductive part of said plant and transmit said location;
e) the at least one camera is in electronic communication with a processor configured to (A) identify a location of a female reproductive part of the at least one plant; and (B) transmit a location signal to a reception unit in response to identifying the location;
f) the preferred temperature is between about 0.5° C. and about 10° C.;
g) the system comprises a conveyor attached to the receptacle configured to facilitate the transfer of the dry pollen to the container, wherein the container comprises a liquid medium;
h) the system comprises a guide head configured to position a plant in an upright position in front of the applicator;
i) the transfer is facilitated by gravity, positive pressure, siphon feeding, a positive displacement pump, a centrifugal pump, or a peristaltic pump; or
j) the pollen is from a cereal plant.

4. The system of claim 3, wherein the applicator is configured to direct the spray of the liquid pollen suspension solution toward said location.

5. The system of claim 4, wherein:
a) the applicator is attached to a repositioning assembly configured to position the applicator in response to receiving said location; or
b) the applicator comprises a plurality of outlets.

6. The system of claim 5, wherein the applicator is configured to variably regulate a flow of the pollen suspension solution from said plurality outlets to direct the spray of the liquid pollen suspension solution toward said location.

6. The system of claim 3, wherein the reception unit is configured to (i) receive the location signal from the identification unit; and (ii) cause at least one applicator from said plurality of applicators to direct the spray of the liquid pollen suspension solution toward the female reproductive part of the at least one plant in response to receiving the location signal.

7. The system of claim 3, wherein said cereal plant is a corn, rice, wheat, or sorghum plant.

8. A method for liquid-mediated delivery of pollen to a recipient plant, the method comprising:
(a) providing the system for liquid-mediated delivery of pollen according to claim 1; and
(b) spraying the liquid pollen suspension solution onto at least a first female reproductive part of the recipient plant using said system, thereby pollinating the recipient plant.

9. The method of claim 8, wherein:
a) the pollen is:
A) from a monocot plant; or
B) recalcitrant pollen; or
b) said liquid pollen suspension is produced less than about 1 hour prior to said spraying:
c) said spraying comprises spraying the liquid pollen suspension solution with a gas pressure of between about 5 psi and about 30 psi;
d) said spraying comprises spraying the liquid pollen suspension solution with an exit velocity of between about 1 m/s and about 10 m/s;
e) said spraying produces droplets with a volume weighted mean droplet diameter of less than about 300 μm;
f) said spraying comprises air-assisted spraying;
g) said method produces a substantially equivalent number of seeds compared to the number of seeds produced using a conventional pollination technique;
h) agitating said liquid pollen suspension prior to or concurrently with said spraying; or
i) the recipient plant is male sterile at the time of said pollinating.

10. The method of claim 9, wherein:
a) the pollen is from a cereal plant;
b) said liquid pollen suspension solution is produced less than about 20 minutes prior to said spraying;
c) the agitating comprises mechanically moving the container; or
d) the agitating comprises sparging the pollen suspension solution with a gas.

11. The method of claim 10, wherein:
a) said cereal plant is a corn, rice, wheat, or sorghum plant; or
b) said liquid pollen suspension is produced less than about 5 minutes prior to said spraying.

12. The method of claim 11, wherein said liquid pollen suspension is produced less than about 30 seconds prior to said spraying.

13. The method of claim 8, further comprising:
a) repeating said steps a) and b) on two or more consecutive days; or
b) collecting seed resulting from said pollinating.

14. The method of claim 13, further comprising crossing a progeny plant grown from said seed with itself or a second plant.

15. The system of claim 1, wherein the system comprises a chamber attached to the container configured to store a liquid medium.

16. The system of claim 15, further comprising a conduit configured to facilitate the transfer of the liquid medium to the container, the conduit comprising a first end and a second end, wherein the first end is connected to the container and the second end is connected to the chamber.

17. The system of claim 16, wherein the transfer is facilitated by gravity, positive pressure, siphon feeding, a positive displacement pump, a centrifugal pump, or a peristaltic pump.
